(19) United States
(12) Patent Application Publication
(10) Pub. No.: US 2025/0170045 A1
(43) Pub. Date: May 29, 2025

(54) HAIR STYLING COMPOSITION

(71) Applicant: Dyson Technology Limited, Malmesbury, Wiltshire (GB)

(72) Inventors: Nathan Charles BROWN, Chippenham (GB); Naomi Mary SIMPSON, Swindon (GB); Charles Robert SMITH, Stroud (GB); Sharon Louise MARTINY, Holybourne (GB)

(21) Appl. No.: 18/842,860

(22) PCT Filed: Mar. 13, 2023
(86) PCT No.: PCT/GB2023/050581

§ 371 Date: Aug. 30, 2024

(30) Foreign Application Priority Data
Mar. 18, 2022 (GB) 2203778.2

(51) Int. Cl. A61K 8/73; A61K 8/06; A61K 8/81; A61Q 5/06

(52) U.S. Cl. CPC A61K 8/736; A61K 8/06; A61K 8/8152; A61Q 5/06

(57) ABSTRACT

A hair styling composition containing chitosan, and a cationic acrylate polymer. The cationic acrylate polymer may contain a 2 methacryloxyethyltrimethylammonium chloride polymer or copolymer, and may be a polyquaternium-37 and/or polyquaternium-32. The combination of chitosan and cationic acrylate polymer provides a hair styling composition with desirable shear-thinning properties, while being sufficiently stable.

The invention relates to a hair styling composition or a composition for a hair styling product.

### BACKGROUND

Hair styling products are commonly used to improve style retention and hold. They are often used prior to styling by heat treatment (such as heated straighteners or curlers). The products may also act to condition or nourish the hair, or protect it from heat. Different hair types have different hair care requirements, and for this reason it has been known to provide different compositions for different hair types, with different active ingredients.

Compositions for hair styling products must include ingredients that impart the required hair care treatments and styling properties. They must also be of an appropriate consistency for the particular application and give a desirable tactile ‘feel’ to the user. Hair styling compositions may be applied in many different formats: for example, as a liquid spray, a foam, a viscous liquid, a balm, a wax, or a cream. Maintaining an easy-to-use consistency is particularly important. The product must coat the hair effectively, but must also disperse easily and evenly. It must also be easily and safely stored in a container. The composition must also be chemically and physically stable.

It is against this background that the invention has been devised.

### STATEMENTS OF INVENTION

The invention resides in a hair styling composition comprising chitosan and a cationic acrylate polymer. The cationic acrylate polymer acts as a stabilising polymer that stabilises the composition despite the presence of the chitosan.

The cationic acrylate polymer may comprise a 2-methacryloxyethyltrimethylammonium chloride polymer or copolymer. Without wishing to be bound by theory, the inventors believe that the 2-methacryloxyethyltrimethylammonium chloride unit is particularly beneficial because of its combination of a large size, and the positive charge helping to stabilise the electron-rich oxygen atoms on the monomers, more specifically the acetylated monomer, of chitosan.

The cationic acrylate polymer may comprise polyquaternium-37 and/or polyquaternium-32.

The hairstyling composition may comprise between approximately 0.15 wt % and approximately 0.45 wt % cationic acrylate polymer, preferably between approximately 0.15 wt % and approximately 0.25 wt %, and most preferably approximately 0.2 wt %. This amount of cationic polymer has been found to be sufficient to stabilised the chitosan, without adversely affecting viscosity of the product.

The composition may comprise less than 0.4 wt % chitosan, preferably between approximately 0.2 wt % and approximately 0.35 wt % chitosan, preferably approximately 0.3 wt %. This low level of chitosan can be stabilised by the stabilising polymer whilst still providing very good hold.

A ratio of chitosan to cationic acrylate polymer is preferably between approximately 4:9 and approximately 8:3, preferably between approximately 4:5 and approximately 7:3, and is most preferably approximately 3:2. These ratios provide sufficient stabilising polymer to stabilise the chitosan.

To aid in stabilisation, the composition may further comprise a chelant. Preferably the chelant is tetrasodium EDTA, Disodium EDTA, Sodium Phytate, Phytic Acid, Tetrasodium Glutamate Diacetate, Trisodium Ethylenediamine Disuccinate. Most preferably the chelant is tetrasodium EDTA, Disodium EDTA or a direct analogue.

The composition may further comprise a viscosity modifier. The viscosity modifier may optionally be one or more of: an alcohol, coconut oil, hydroxyethyl cellulose, guar gum, castorlatum, or shea butter. The viscosity modifier can further tune the viscosity to provide an optimised viscosity, and in particular a shear-thinning viscosity. The viscosity modifier preferably comprises a long-chain alcohol, which most preferably comprises at least 14 carbon atoms in the chain.

The viscosity modifier may comprise stearyl alcohol and/or cetearyl alcohol and/or cetyl alcohol. The hair styling product may comprise between approximately 2.5 wt % and approximately 3.5 wt % stearyl alcohol and/or cetearyl alcohol and/or cetyl alcohol. The viscosity modifier may comprise a blend of alcohols, and the blend may comprise for example behenyl alcohol.

The hair styling composition may further comprise one or more emulsifiers. The emulsifier may be a stearate, preferably glyceryl stearate and/or PEG-100 stearate. Preferably the hair styling product comprises between approximately 0.5 wt % and approximately 1.5 wt % stearate.

The composition may comprise for example between 0.5 wt % and 3.0 wt % stearate and/or polyglycerol in combination, and preferably between 0.8 wt % and 2.5 wt % stearate and/or polyglycerol in combination.

The hair styling composition may comprise a solvent for plasticising the chitosan. The solvent may comprise for example butylene glycol, Propylene glycol, glycerin, or panthenol.

To provide a hair conditioning function, the hair styling composition may comprise one or more conditioning components. The conditioning components may optionally include one or more oils, gums or polymers.

The hair styling composition is preferably a shear-thinning fluid. In rheology measurements as measured by a rotational rheometer (Anton Parr MCR302, steady state shear rate ramp, 25 mm sand-blasted parallel plates, 25° C.), the hair styling product preferably displays a viscosity of at least 1.25×10⁵ cP at a frequency of 0.1 s⁻¹, and less than 1500 cP at a frequency of 100 s⁻¹. Most preferably the hair styling product displays a viscosity of at least 1.5×10⁵ cP at a frequency of 0.1 s⁻¹, and or a viscosity of less than 1200 cP at a frequency of 100 s⁻¹.

### DESCRIPTION

The hair styling composition described herein combines particularly good hold properties, with a smooth and easy application. As a result, the composition can be applied evenly, which avoids a ‘crunchy’ texture and provides a natural feel.

The particularly good hold properties are achieved by use of a chitosan. Chitosan provides excellent hold, but is difficult to stabilise in a composition, particularly in high quantities, and particularly in combination with oils that might be required for other functions (such as hair conditioning). The inventors have found that small quantities of chitosan can be stabilised using a stabilising polymer, in particular a cationic acrylate polymer, and will still provide excellent hold.

The inventors have found that a particularly effective stabilising polymer is a polymer that comprises units of 2-methacryloxyethyltrimethylammonium chloride, as either a polymer or co-polymer. Examples are polyquaternium 37 and polyquaternium 32. An example of a particularly suitable polymer is for example Cosmedia Ultragel 300 (polyquaternium 37), which is commercially available from BASF®.

The combination of chitosan and cationic acrylate polymer provides a particularly stable composition that gives good hold.

The chitosan is provided in a particularly small amount: for example between approximately 0.2 wt % of the entire composition and approximately 0.4 wt % of the entire composition. Preferably the composition comprises between approximately 0.2 wt % and approximately 0.35 wt % chitosan, preferably approximately 0.3 wt %. Larger amounts of chitosan are difficult to stabilise, whereas smaller amounts may not provide sufficient hold. The inventors have surprisingly found that the small amounts described above provide very good hold, whilst being stabilised by the stabilising polymer.

The chitosan may be derived from any suitable source. In one preferred example the chitosan is mushroom-derived. A suitable chitosan is for example Vida-Care KytoStyle V, commercially available from KCC Basildon (Momentive).

The stabilising polymer is also provided in a small amount: for example between 0.15 wt % and approximately 0.45 wt %, preferably between approximately 0.15 wt % and approximately 0.25 wt %, and most preferably approximately 0.2 wt %.

A particularly preferred ratio of the chitosan to the stabilising polymer by weight is between approximately 4:9 and approximately 8:3, preferably between approximately 4:5 and approximately 7:3, and is most preferably approximately 3:2.

To optimise the viscosity properties, one or more viscosity modifiers may be included the composition, which may comprise one or more of the following components:
1) An alcohol. An alcohol may be included to act as a rheology additive. Particularly preferred alcohols are stearyl alcohol and/or cetearyl alcohol and/or cetyl alcohol. Stearyl alcohol may provide greater thermal stability, and may therefore be preferred if the composition contains otherwise destabilising ingredients. The composition preferably comprises between approximately 2% and approximately 4% alcohol, and most preferably between approximately 2.5% and approximately 3.5% alcohol. A blend of alcohols may be used, and the blend may comprise for example behenyl alcohol. The alcohol is preferably a long-chain alcohol, and preferably comprises at least 14 carbon atoms in the chain.
2) Coconut oil. A coconut oil may be used as a thickener. The composition may comprise for example between 1 wt % and 4 wt % coconut oil, and preferably between 1.5 wt % and 3.5 wt % coconut oil.
3) One or more emulsifiers, such as a stearate, or a polyglycerol. Suitable stearates are glyceryl stearate and/or polyethylene glycol stearate (which may for example be PEG-100 stearate), and preferably a mixture of both. Suitable polyglycerols are for example Polyglyceryl-6 distearate and/or Polyglyceryl-3 beeswax. The polyglycerols may be provided as part of a mixture comprising other components: for example as Emulium Mellifera MB available from GATTEFOSSE. The composition may comprise for example between 0.5 wt % and 3.0 wt % stearate and/or polyglycerol in combination, and preferably between 0.8 wt % and 2.5 wt % stearate and/or polyglycerol in combination.

Other components with an emulsifying function may be used, including for example C12-14 sec-Pareth-9 & C12-14 sec-Pareth-5.
4) Hydroxyethyl cellulose. Hydroxyethyl cellulose may be used to increase the viscosity of the composition. The composition may comprise between 0.25 wt % and 1.5 wt % hydroxyethyl cellulose, end most preferably comprises between 0.8 wt % and 1.2 wt % hydroxyethyl cellulose.

By virtue of these viscosity modifying components, particularly desirable viscosity properties can be achieved, while still retaining a stable product and good hold. The chitosan, stabilising polymer and viscosity modifying components act in synergy to provide a balance of desired properties.

The inventors have found that a particularly desirable viscosity profile, as measured by a rotational rheometer (Anton Parr MCR302, steady state shear rate ramp, 25 mm sand-blasted parallel plates, 25° C.), is at least 1.25×10⁵ cP at a frequency of 0.1 s⁻¹, and less than 1500 cP at a frequency of 100 s⁻¹. This viscosity behaviour is shear-thinning (i.e, with a high viscosity at low frequencies and a low viscosity at high frequencies), so that the composition has the texture of thick cream when stored in a container and scooped out for use, but a thin and slippery consistency when spread onto the hair, so that it can be easily and evenly distributed.

An even more particularly desirable viscosity profile is at least 1.5×10⁵ cP at a frequency of 0.1 s⁻¹, and or a viscosity of less than 1200 cP at a frequency of 100 s⁻¹.

The composition may comprise other components including:
1) A solvent for plasticising the chitosan and other components of the composition. A suitable example is a butylene glycol, such as 1,3 butylene glycol. Other examples include Propylene Glycol, Glycerin, and Panthenol. The composition may comprise between 0.25 wt % and 1.5 wt % solvent, and most preferably comprises between 0.8 wt % and 1.2 wt % solvent.
2) A chelant, for providing additional stability. A suitable chelant is for example tetrasodium EDTA. The composition may comprise for example between 0.02 wt % and 0.1 wt % chelant, preferably between 0.4 wt % and 0.06 wt % chelant. Other suitable chelants include Disodium EDTA, Sodium Phytate, Phytic Acid, Tetrasodium Glutamate Diacetate, and Trisodium Ethylenediamine Disuccinate.
3) A pH controller, such as an acid for lowering the pH. A suitable acid is lactic acid, for example a lactic acid at 88% concentration in water. The composition may comprise an amount of lactic acid that is necessary to achieve the desired pH, for example between 0.1 wt % and 0.5 wt %, preferably between 0.2 wt % and 0.25 wt %. Other suitable pH controllers include citric acid. Preferably the pH of the formulation is less than 5.
4) One or more preservatives. Suitable preservatives may comprise for example, phenoxyethanol, Caprylyl Glycol, benzoic acid and/or dehydroacetic acid. The preservatives may be provided as commercially available preservative mixtures, such as Optiphen™ and/or Rokonsal™ that are commercially available from Ashland®. The composition may comprise for example between 0.5 wt % and 2.0 wt % preservative, preferably between 1 wt % and 1.5 wt %.
5) One or more hair conditioning components. Suitable hair conditioners include oils, gums and polymers, such as Behenyltrimonium Chloride, C13-15 alkane, dimethicone, diphenyl dimethicone, oils such as Vitis Vinifera (Grape) Seed Oil Castor Oil (hydrogenated and/or unhydrogenated), Cocos Nucifera (Coconut) Oil, Argania Spinosa (Argan) Kernel Oil, Ricinus Communis Seed Oil, Hydrogenated Castor Oil. The composition may comprise for example between 5 wt % and 25 wt % conditioning components, and preferably comprises between 10 wt % and 20 wt % conditioning components.
6) Fragrance or parfum, which may be any suitable fragrance. The composition may comprise for example between 0.05 wt % and 1 wt % fragrance, and preferably comprises between 0.1 wt % and 0.5 wt % fragrance.

The composition also comprises a suitable carrier or solvent, which is typically water. This carrier makes up the balance of the composition.

The additional ingredients, and the relative quantities of those ingredients, can be varied to make different compositions that are suitable for different hair types. For example, for a coarser hair type, a higher content of conditioning components may be used, and/or different conditioning components may be used. Since this will affect the viscosity of the composition, different quantities of viscosity modifiers may be required, and/or different viscosity modifying components may be needed, to achieve a particularly preferred viscosity.

To make the composition, the components are mixed together using to standard emulsion mixing procedures. Specifically, appropriate combinations of the components are pre mixed in appropriate vessels as pre-mix phases. Different pre-mix phases are then added to a main vessel in an appropriate order, with mixing as each pre-mix phase is added. The mixture(s) may be heated and cooled for mixing if appropriate.

### EXAMPLES

### Example 1

Samples of hair care formulation were made to compare a control Sample 0 against Samples 1 to 6. The sample ingredients were selected in particular to compare the effect of the following variables in the composition compared to control Sample 0:
Sample 1 Increased PQ37
Sample 2 Lower Chitosan
Sample 3 Higher Chitosan
Sample 4 Higher Chitosan
Sample 5 No PQ37 or PQ32
Sample 6 PQ32 instead of PQ37

The ingredients of Sample 0 and Samples 1 to 6 were as indicated in Table 1 below:

TABLE 1

| | | Sample Number | | | | | | |
|---|---|---|---|---|---|---|---|---|
| | | 0 | 1 | 2 | 3 | 4 | 5 | 6 |
| Trade Name | INCI Name | % w/w | | | | | | |
| Deionised | Aqua | 83.33 | 82.53 | 83.75 | 83.70 | 83.75 | 83.53 | 83.53 |
| Water | | | | | | | | |
| Na₄ EDTA | Tetrasodium | 0.05 | 0.05 | 0.05 | 0.05 | 0.05 | 0.05 | 0.05 |
| | EDTA | | | | | | | |
| Vida-Care HEC | Hydroxyethyl | 0.50 | 0.50 | 0.50 | 0.50 | 0.50 | 0.50 | 0.50 |
| | Cellulose | | | | | | | |
| Cosmedia | Polyquaternium | 0.20 | 1.00 | 0.20 | 0.20 | 0.20 | n/a | n/a |
| Ultragel 300 | 37 | | | | | | | |
| Salcare SC 92 | Polyquaternium- | n/a | n/a | n/a | n/a | n/a | n/a | 0.20 |
| | 32 (and) Mineral | | | | | | | |
| | Oil (and) PPG-1 | | | | | | | |
| | Trideceth-6 | | | | | | | |
| Butylene Glycol | Butylene Glycol | 1.00 | 1.00 | 1.00 | 1.00 | 1.00 | 1.00 | 1.00 |
| Optiphen | Phenoxyethanol | 1.00 | 1.00 | 1.00 | 1.00 | 1.00 | 1.00 | 1.00 |
| | (and) Caprylyl | | | | | | | |
| | Glycol | | | | | | | |
| Vida-Care | Chitosan | 0.30 | 0.30 | 0.20 | 0.35 | 0.40 | 0.30 | 0.30 |
| Kytostyle-V | | | | | | | | |
| Lactic Acid | Aqua (and) | 0.22 | 0.22 | 0.15 | 0.26 | 0.29 | 0.22 | 0.22 |
| 80% Aq | Lactic Acid | | | | | | | |
| Grapeseed Oil | Vitis Vinifera | 3.80 | 3.80 | 3.80 | 3.80 | 3.80 | 3.80 | 3.80 |
| | (Grape) Seed | | | | | | | |
| | Oil | | | | | | | |
| Coconut Oil | Cocos Nucifera | 3.40 | 3.40 | 3.40 | 3.40 | 3.40 | 3.40 | 3.40 |
| | (Coconut) Oil | | | | | | | |
| Sabowax FL | Glyceryl | 1.00 | 1.00 | 1.00 | 1.00 | 1.00 | 1.00 | 1.00 |
| 65K | Stearate (&) | | | | | | | |
| | PEG-100 | | | | | | | |
| | Stearate | | | | | | | |
| Crodacol CS | Cetearyl Alcohol | 3.00 | 3.00 | 3.00 | 3.00 | 3.00 | 3.00 | 3.00 |
| 90 | | | | | | | | |
| SeraSense | C13-15 alkane | 2.00 | 2.00 | 2.00 | 2.00 | 2.00 | 2.00 | 2.00 |
| GBA 81 | (and) | | | | | | | |
| | Dimethicone | | | | | | | |
| Fragrance | Parfum | 0.20 | 0.20 | 0.20 | 0.20 | 0.20 | 0.20 | 0.20 |

To make the compositions, the ingredients were divided into the following phase mixtures shown in Table 2.

TABLE 2

| | |
|---|---|
| A | Deionised Water |
| | Tetrasodium EDTA |
| | Hydroxyethyl Cellulose |
| B | Polyquaternium 37 (if present) |
| | Polyquaternium-32 (and) Mineral Oil |
| | (and) PPG-1 Trideceth-6 (if present) |
| | Butylene Glycol |
| | Phenoxyethanol (and) Caprylyl Glycol |
| C | Deionised Water |
| | Chitosan |
| | Aqua (and) Lactic Acid |
| D | Vitis Vinifera (Grape) Seed Oil |
| | Cocos Nucifera (Coconut) Oil |
| | Glyceryl Stearate (&) PEG-100 Stearate |
| | Cetearyl Alcohol |
| E | C13-15 alkane (and) Dimethicone |
| F | Parfum |

The compositions were then made in a batch process according to the following method shown in Table 3:

TABLE 3

| | |
|---|---|
| 1 | Add water to main mixing vessel and begin stirring with main stirrer |
| 2 | Add phase A materials to the water in the main mixing vessel |
| | Homogenise for 5 minutes. |
| 3 | Heat batch to 65-70° C. |
| | Add phase B materials into main vessel. |
| | Mix until all materials are fully dissolved. |
| 4 | In a separate vessel premix phase C materials together. |
| | Add the Lactic Acid in increments until pH spec of 4.1-4.4 |
| | is met. Continue mixing for a minimum of 10 minutes to ensure |
| | all components are fully hydrolysed/dissolved. |
| 5 | Add phase C premix to main vessel (containing A and B). |
| 6 | In a separate vessel premix phase D |
| | Heat to 65-70° C. whilst stirring until all waxes are melted |
| 7 | Ensure Premix D and main vessel (A, B, C) are both at target |
| | temperature (65-70° C.). |
| | Add Phase D to main vessel, while mixing. |
| | Homogenise batch |
| 8 | Start cooling to ambient temperature, with continued mixing |
| | until composition is uniform. |
| 9 | Add phase E to batch while batch is below 70° C., with mixing |
| | until uniform. |
| 10 | Add phase F to batch while batch is below 40° C., with mixing |
| | until uniform. |

Viscosity tests and high temperature stability tests were conducted on all samples with the following results shown in Table 4.

TABLE 4

| | Sample Number | | | | | | |
|---|---|---|---|---|---|---|---|
| Test | 0 | 1 | 2 | 3 | 4 | 5 | 6 |
| Low Shear | 1.25 × 10⁵ | 5.23 × 10⁵ | 1.34 × 10⁵ | 1.51 × 10⁵ | 8.25 × 10⁴ | 4.02 × 10⁴ | 1.79 × 10⁵ |
| Viscosity- | | | | | | | |
| 0.1 s⁻¹/cP | | | | | | | |
| High Shear | 856 | 2410 | 813 | 962 | 848 | 540 | 971 |
| Viscosity- | | | | | | | |
| 100 s-1/ | | | | | | | |
| cP | | | | | | | |
| High Temp | 2 Weeks | 4 Weeks | 2 Weeks | 2 Weeks | 1 Week | 3 Days | 2 Weeks |
| Stability- | | | | | | | |
| 50° C. | | | | | | | |

* High temperature stability test stopped at 4 weeks.

All samples were found to exhibit shear-thinning properties, with a high viscosity at low frequencies/shear, and a low viscosity at high frequencies/shear, as indicated by the viscosity measurements. The results also show that:
Presence of the cationic acrylate polymer (i.e. the PQ37 or PQ32 in these samples, which is present in all samples other than Sample 5) increased the stability of the formulation.
Formulations with higher levels of chitosan exhibit poorer stability and low shear viscosity.
Formulations with higher levels of the cationic acrylate polymer have greater stability but higher viscosity
Of the Samples tested, Samples 0, 2 and 3 exhibit the most desirable balance of stability and viscosity profile.

The hold was found to be excellent for all samples containing chitosan.

### Example 2

Further samples of hair care formulation were made with ingredients as indicated in Table 5 below. Ingredients were varied so as to test substantially the same formulation with and without the cationic acrylate polymer PQ37.

TABLE 5

| | | Sample | Sample |
|---|---|---|---|
| | | A | B |
| Trade Name | INCI Name | % w/w | % w/w |
| Deionised Water | Aqua | 82.10 | 81.90 |
| Na₄ EDTA | Tetrasodium EDTA | 0.05 | 0.05 |
| Vida-Care HEC | Hydroxyethyl Cellulose | 0.50 | 0.50 |
| Butylene Glycol | Butylene Glycol | 1.00 | 1.00 |
| Optiphen | Phenoxyethanol (and) Caprylyl | 0.75 | 0.75 |
| | Glycol | | |
| Genamin BTLF | Behenyltrimonium Chloride | 0.25 | 0.25 |
| Vida-Care | Chitosan | 0.30 | 0.30 |
| Kytostyle-V | | | |
| Cosmedia | Polyquaternium 37 | | 0.20 |
| Ultragel 300 | | | |
| Optiphen ND | Phenoxyethanol (and) Sodium | 0.30 | 0.30 |
| | Benzoate (and) Dehydroacetic | | |
| | Acid | | |
| Lactic Acid | Aqua (and) Lactic Acid | 0.30 | 0.30 |
| 80% Aq | | | |
| Grapeseed Oil | Vitis Vinifera (Grape) Seed Oil | 2.00 | 2.00 |
| Castorlatum | Hydrogenated Castor Oil (and) | 1.50 | 1.50 |
| | Castor Oil | | |
| Coconut Oil | Cocos Nucifera (Coconut) Oil | 1.50 | 1.50 |
| Argan Oil | Argania Spinosa (Argan) | 2.00 | 2.00 |
| | Kernel Oil | | |
| Sabowax | Glyceryl Stearate (&) PEG-100 | 1.00 | 1.00 |
| FL 65K | Stearate | | |
| Natragem | Polyglyceryl-4 Laurate/Sebacate | 0.50 | 0.50 |
| S150 NP | (&) Polyglyceryl-4 | | |
| | Caprylate/Caprate (&) Aqua | | |
| Crodacol | Cetearyl Alcohol | 2.75 | 2.75 |
| CS 90 | | | |
| Emulium | Polyglyceryl-6 distearate & | 1.00 | 1.00 |
| Mellifera | Jojoba esters & Polyglyceryl-3 | | |
| | beeswax & Cetyl alcohol | | |
| SeraSense | C13-15 alkane (and) Dimethicone | 2.00 | 2.00 |
| GBA 81 | | | |
| Fragrance | Parfum | 0.20 | 0.20 |

To make the compositions, the ingredients were divided into the following phase mixtures shown in Table 6, and the process of Table 3 above was followed.

| | |
|---|---|
| A | Deionised Water |
| | Tetrasodium EDTA |
| | Hydroxyethyl Cellulose |
| B | Polyquaternium 37 |
| | Butylene Glycol |
| | Phenoxyethanol (and) Caprylyl Glycol |
| | Behenyltrimonium Chloride |
| | Polyglyceryl-4 Laurate/Sebacate (&) Polyglyceryl-4 |
| | Caprylate/Caprate (&) Aqua |
| C | Deionised Water |
| | Chitosan |
| | Phenoxyethanol (and) Sodium Benzoate (and) Dehydroacetic Acid |
| | Aqua (and) Lactic Acid |
| D | Vitis Vinifera (Grape) Seed Oil (if present) |
| | Hydrogenated Castor Oil (and) Castor Oil |
| | Cocos Nucifera (Coconut) Oil |
| | Argania Spinosa (Argan) Kernel Oil |
| | Glyceryl Stearate (&) PEG-100 Stearate |
| | Cetearyl Alcohol |
| | Polyglyceryl-6 distearate & Jojoba esters & Polyglyceryl-3 |
| | beeswax & Cetyl alcohol |
| E | C13-15 alkane (and) Dimethicone |
| F | Parfum |

Viscosity tests and high temperature stability tests were conducted on Samples A and B with the results shown in Table 7 below.

TABLE 7

| | | | Sample | Sample |
|---|---|---|---|---|
| Test | Detail | | A | B |
| Low Shear Viscosity | 0.1 | s⁻¹/cP | 1.08 × 10⁵ | 1.27 × 10⁵ |
| High Shear Viscosity | 100 | s − 1/cP | 726 | 1107 |
| Accelerated Stability | 40° | C. | 1 Week | 8 Weeks |
| High Temp Stability | 50° | C. | 1 Week | 4 Weeks |

* High temperature stability test stopped at 4 weeks.

The results show that addition of the cationic acrylate polymer significantly increases the low shear viscosity, whilst having minimal impact on the high shear viscosity. The stability is also significantly improved. This improves the handleability of the formula whilst also significantly, and unexpectantly, improving the stability of the formulations.

### Example 3

Styling performance tests were conducted by applying Sample B of Example 2 to towel dried tresses (6 g, 35 cm long, 2.5 cm biding width, untreated European origin fine hair) and styling the tresses using a Dyson Airwrap with the 30 mm curling attachment. 0.35 g of product was applied to the Sample B tresses whilst the hair is damp and massaged evenly across the tress before styling. A control sample was styled in the same way, with no product applied.

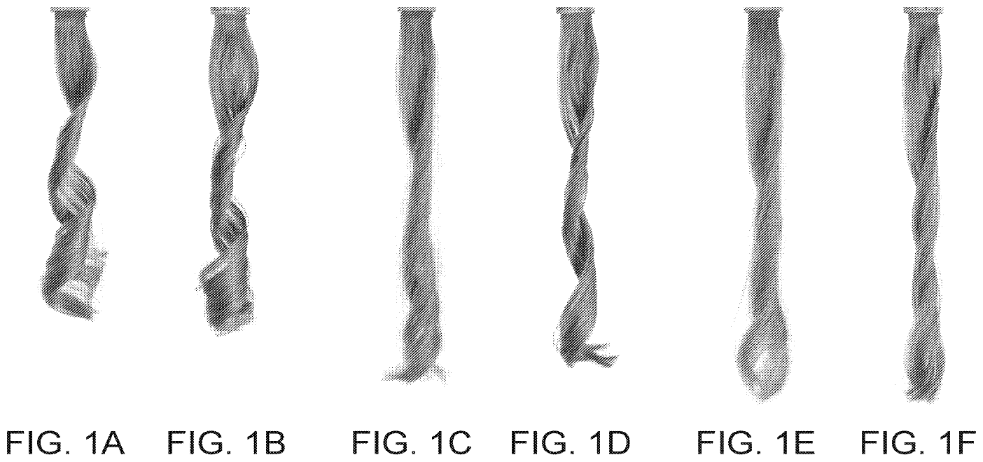

Images were taken immediately after styling then after 1 hour and after 24 hours in a 80% RH/25° C. humidity chamber. Images are shown in FIGS. 1A to 1F, and Table 8 below indicates the product applied and time since styling for each figure.

8

| FIG. | Product applied | Time since styling |
|---|---|---|
| 1A | None | Immediately after styling |
| 1B | Sample B | Immediately after styling |
| 1C | None | 1 hr at 80% RH |
| 1D | Sample B | 1 hr at 80% RH |
| 1E | None | 24 hrs at 80% RH |
| 1F | Sample B | 24 hrs at 80% RH |

As can be seen in the Figures, curl quality/structure is more defined for the tresses with product applied, with less frizz and fly-aways at all time points, even after significant exposure to high humidity conditions.

1. A hair styling composition comprising:
chitosan; and
a cationic acrylate polymer.

2. The hair styling composition of claim 1, wherein the cationic acrylate polymer comprises a 2-methacryloxyethyltrimethylammonium chloride polymer or copolymer.

3. The hair styling composition of claim 2, wherein the cationic acrylate polymer comprises polyquaternium-37 and/or polyquaternium-32.

4. The hair styling composition of claim 1, comprising between approximately 0.15 wt % and approximately 0.45 wt % cationic acrylate polymer.

5. The hair styling composition of claim 1, comprising less than 0.4 wt % chitosan.

6. The hair styling composition of claim 1, wherein a ratio of chitosan to cationic acrylate polymer is between approximately 4:9 and approximately 8:3.

7. The hair styling composition of claim 1, further comprising a chelant.

8. The hair styling composition of claim 1, further comprising a viscosity modifier.

9. The hair styling composition of claim 8, wherein the viscosity modifier comprises an alcohol.

10. The hair styling composition of claim 9, wherein the viscosity modifier comprises stearyl alcohol and/or cetearyl alcohol and/or cetyl alcohol.

11. The hair styling composition of claim 10, wherein the hair styling composition comprises between approximately 2.5 wt % and approximately 3.5 wt % stearyl alcohol and/or cetearyl alcohol and/or cetyl alcohol.

12. The hair styling composition of claim 1, further comprising one or more emulsifiers.

13. The hair styling composition of claim 12, wherein the one or more emulsifiers comprise a stearate.

14. The hair styling composition of claim 13, wherein the composition comprises between 0.5 wt % and 3.0 wt % emulsifier.

15. The hair styling composition of claim 1, comprising a solvent for plasticising the chitosan.

16. The hair styling composition of claim 1, comprising conditioning components.

17. The hair styling composition of claim 1, wherein the hair styling composition is a shear-thinning fluid.

18. The hair styling composition of claim 1, having a viscosity as measured by a rotational rheometer (Anton Parr MCR302, steady state shear rate ramp, 25 mm sand-blasted parallel plates, 25° C.) of at least 1.25×10⁵ cP at a frequency of 0.1 s⁻¹, and less than 1500 cP at a frequency of 100 s⁻¹.

19. The hair styling composition of claim 18, having a viscosity as measured by a rotational rheometer (Anton Parr MCR302, steady state shear rate ramp, 25 mm sand-blasted parallel plates, 25° C.) of at least 1.5×10⁵ cP at a frequency of 0.1 s⁻¹, and a viscosity of less than 1200 cP at a frequency of 100 s⁻¹.

20. The hair styling composition of claim 1, having a pH of less than 5.
